(19) United States
(12) Patent Application Publication
(10) Pub. No.: US 2002/0022217 A1
(43) Pub. Date: Feb. 21, 2002

(54) HIGH DENSITY STREPTAVIDIN SUPPORTS

(76) Inventors: CHANDRAN R. SABANAYAGAM, ALLSTON, MA (US); CHARLES R. CANTOR, BOSTON, MA (US); CASSANDRA L. SMITH, BOSTON, MA (US)

Correspondence Address: RONALD I. EISENSTIEN, NIXON PEABODY LLP, 101 FEDERAL STREET, BOSTON, MA, 02110, US

(21) Appl. No.: 09/104,100

(22) Filed: Jun. 24, 1998

Related U.S. Application Data
(60) Provisional application No. 60/050,662, filed on Jun. 24, 1997.

(51) Int. Cl. C12Q 1/00; C12Q 1/68; G01N 33/53; G01N 33/543

(52) U.S. Cl. 435/4; 435/6; 435/7.1; 435/7.5; 438/1

(57) ABSTRACT

The coating of silicon surfaces is described, including but not limited to coating silicon surfaces with biomolecules such as biotin and avidin. Silicon surfaces are made with a monolayer of the protein streptavidin and these surfaces are then used as supports for reactions, including but not limited to nucleic acid hybridization.

This application for patent under 35 U.S.C. 111(a) claims priority to Provisional Application Ser. No. 60/050,662, filed Jun. 24, 1997 under 35 U.S.C. 111(b). This invention was made with Government Support under Contract Number DE-FG02-93ER61656 awarded by the Department of Energy. The government has certain rights in the invention.

### FIELD OF THE INVENTION

The present invention relates to the coating of silicon surfaces, including but not limited to coating silicon surfaces with biomolecules such as biotin and avidin.

### BACKGROUND

Identification of specific biomolecules (e.g. nucleic acids and proteins) is critical to many aspects of medicine and molecular biology, for instance, diagnosis of diseases, infections or genetic mutations. Conventional approaches typically are based upon immunological methods (e.g. antibody assays), histological methods (e.g. cellular morphology assays), or direct identification of nucleic acid sequences (e.g. hybridization or DNA sequencing).

With regard to immunological methods, solid-state immunoassays utilize specific antibodies bound to a polymeric support (polyvinylchloride) to detect a specific protein (antigen) produced by a pathogen. Cellular extracts (serum or urine) are placed directly on the support to facilitate detection. By this method, typically one nanogram of antigen can be reliably detected.

Histologic approaches utilize microscopic (light or election microscopy) examination of a tissue sample (biopsy) to identify diseased cells by their morphological differences from normal cells. Histologic approaches are slow and typically only provide a qualitative result (in contrast to other methods that can provide quantitative results).

The polymerase chain reaction (PCR) has facilitated rapid and accurate detection of pathogens by direct amplification of trace amounts of viral or bacterial DNA present in blood or tissue samples. While PCR is powerful, however, is complicated by carryover problems.

Regardless of the type of assay used, assays in the past have typically been carried out in single sample formats. For large scale analysis, such formats are not practical.

New formats are being explored that permit multiple samples to be assayed simultaneously. One such format is the “high-density array.” See M. Kozal et al., “Extensive polymorphisms observed in HIV-1 clade B protease gene using high-density oligonucleotide arrays,” Nature Medicine 2:753 (1996). See also D. Shalon et al., “A DNA Microarray System for Analyzing Complex DNA samples Using Two-color Fluorescent Probe Hybridization,” Genome Res. 639-645 (1996). Use of numerous (100s-1,000s) immobilized probes (antibodies or nucleic acids) on a single biochip has the potential to greatly facilitate pathogen and genetic screenings. Also, because of the massive parallel processing inherent to a biochip, much less sample is required to screen for the 100s-1,000s pathogens. Furthermore, because analysis of these biochips are performed using microscopes, the sensitivity of detection is enhanced through optical magnification.

Unfortunately, the manufacture of high density arrays is complicated. Currently used chemistries can make the manufacturing costly and unreliable. What is needed is an improved approach to the manufacture of such arrays that permits inexpensive and reliable array fabrication.

### SUMMARY OF THE INVENTION

The present invention relates to the coating of silicon surfaces, including but not limited to coating silicon surfaces with biomolecules such as biotin and avidin. The coating methods, chemistries and compositions of the present invention permits a new approach to the manufacture of high density arrays on biochips. In brief the approach involves the covalent attachment of biotin to the appropriately modified surface of a solid support (e.g. silanized silicon dioxide), followed by the non-covalent attachment of streptavidin (or other suitable avidin analog) to the surface bound biotins. Thereafter, a variety of biotin-linked molecules can be attached to the solid support (by virtue of the fact that streptavidin is multivalent), including but not limited to biotin-linked antibodies, biotin-linked single-stranded and double-stranded nucleic acids, and biotin-linked protein (shown schematically in FIG. 1).

It is not intended that the present invention be limited to the particular method by which biotin is attached to a silanized surface. In one embodiment, biotin is photochemically attached. In another embodiment, biotin is chemically attached.

When photochemical attachment is used, the position of biotin on the surface of the solid support (and consequently the position of streptavidin and other biomolecules) can be controlled or “patterned.” In one embodiment, such patterning is achieved by the use of a photomask and photoactivatible biotin. As shown schematically in FIG. 2, using the proper wavelength of light (e.g. 365 nm), the photoactivatible biotin will be linked to the support surface where the photomask does not block the light. Labelled streptavidin can be used to readily detect (and quality control) the patterning of the biotin.

Patterning can also be carried out without a photomask. For example, any method that selectively directs the proper wavelength of light (e.g. a method involving a laser) can be used to pattern the position of the photochemically linked biotin.

It is also not intended that the present invention be limited to a particular chemical means of attachment. In this regard, the biotin molecule can be thought of as a molecule having three parts: biotin-linker-X, where X is the reactive group. The surface of the solid support can also be characterized in three parts: surface-linker-Y, wherein Y is a functional group capable of chemically interacting with X.

Y can be a variety of functional groups, including but not limited to a primary amine group (—NH₂), a sulfhydryl group (—SH), a carboxyl group (—COOH) or an oxidized carbohydrate (—COH). Where Y is a primary amine, X is conveniently an NHS-ester or Sulfo-NHS-ester (a variety of such modified biotin molecules are available commercially from Pierce Chemical Co., Rockford, Ill.). Where Y is a sulfhydryl group, X is conveniently a maleimide or iodoacetyl group (a variety of these biotin molecules are also available commercially from Pierce Chemical Co., Rockford, Ill.). Where Y is an oxidized carbohydrate, X is conveniently a hydazide (a variety of such biotin-hydrazide molecules are available commercially from Pierce Chemical Co., Rockford, Ill.).

The present invention contemplates a composition, comprising biotin covalently attached to (e.g. immobilized on) a solid support, said solid support comprising silicon dioxide and said biotin non-covalently attached to streptavidin. In a preferred embodiment, said biotin is attached to only a portion of said solid support (i.e. the biotin is patterned); for example, biotin is attached in a discrete region of the solid support and not present in other regions of the solid support.

The present invention also contemplates a method, comprising a) providing i) biotin covalently attached to (e.g. immobilized on) a solid support, said solid support comprising silicon dioxide, and ii) streptavidin; and b) non-covalently attaching said streptavidin to said biotin, thereby attaching said streptavidin to said solid support. In one embodiment, the method further comprises the step of binding a biotin-linked biomolecule to said streptavidin attached to said solid support. In a preferred embodiment, said biotin-linked biomolecule is selected from the group consisting of biotin-linked proteins and biotin-linked nucleic acids.

### DESCRIPTION OF THE DRAWINGS

FIG. 1 schematically shows biotin covalently attached to (e.g. immobilized on) a solid support, said solid support comprising silicon dioxide (SiO₂) and said biotin non-covalently attached to streptavidin, said streptavidin forming a monolayer and being bound to a variety of biotin-linked biomolecules (a biotin-linked antibody is shown on the far left, while biotin-linked single- and double-stranded nucleic acid is shown in the middle, and biotin-linked carbohydrate is represented on the far right).

FIG. 2 schematically shows one embodiment of the method of the present invention for attaching biotin, wherein photoactivatible biotin is linked to the support surface where the photomask (shown as solid black rectangles) does not block the light (shown as arrows).

FIG. 3 shows a representative silanization reaction.

FIGS. 4A, 4B, 4C and 4D schematically show silanization followed by attachment of biotin to a silicon support.

FIGS. 5A, 5B and 5C are micrographs of several patterned streptavidin surfaces.

FIG. 6 is a graph showing the free biotin-binding capacity of the streptavidin-coated supports of the present invention (“Cs”=surface concentration).

FIG. 7 is a graph showing the binding of biotinylated nucleic acid to the streptavidin-coated supports of the present invention.

FIGS. 8A and 8B schematically show the top view and side view of a microwell etched in silicon. FIG. 8C is a micrograph showing streptavidin patterned in a silicon microwell.

FIG. 9 is an autoradiogram of ³²P-labeled biotinylated oligonucleotides immobilized in streptavidin-coated silicon microwells (wells outside the dotted box are controls used for quantitation).

FIG. 10 is a graph showing the immobilization of oligonucleotides in the context of concentration and length.

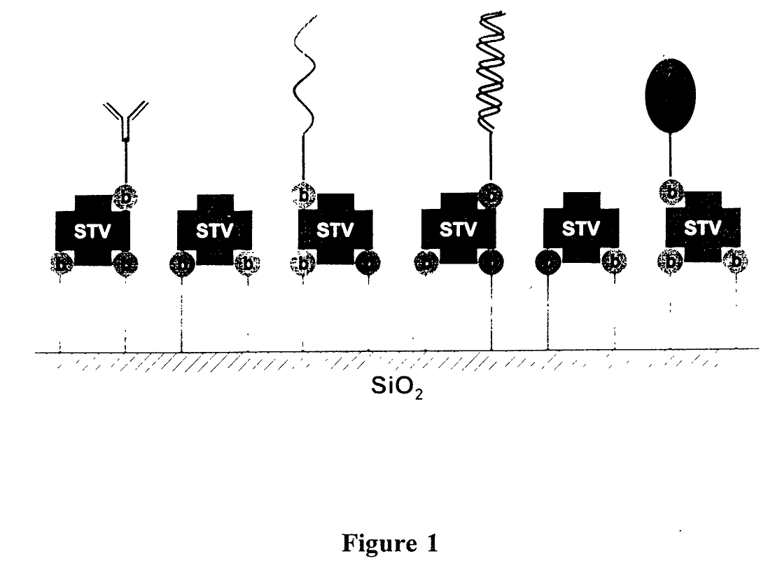

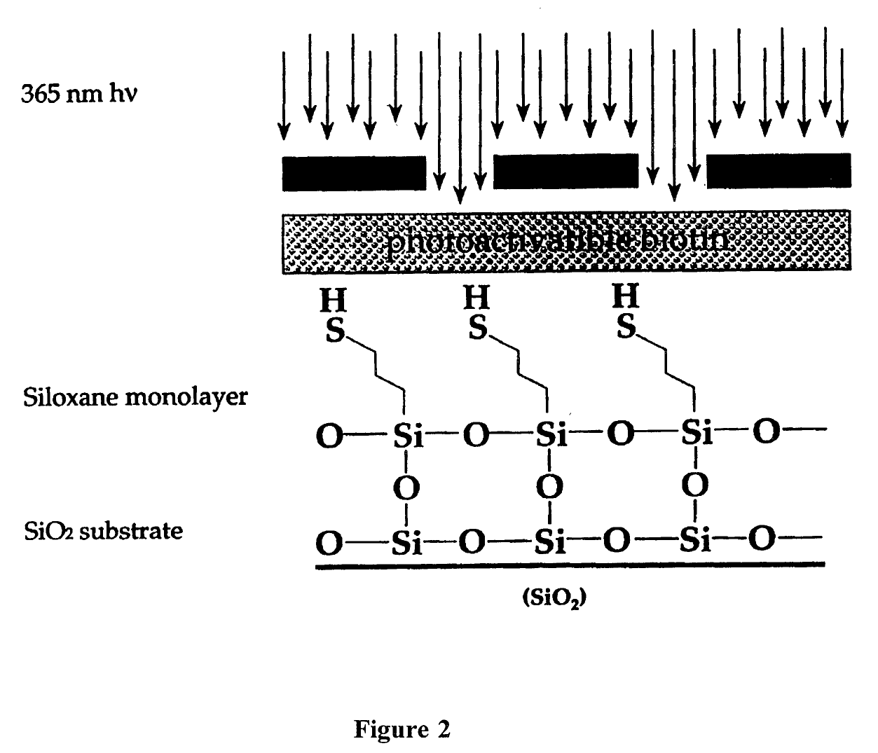

### DEFINITIONS

To facilitate understanding of the invention, a number of terms are defined below.

The term “biomolecules” includes proteins (e.g. enzymes, antibodies etc.), carbohydrates, lipids, nucleic acids (both RNA and DNA) and nucleic acid analogs (such as PNA).

The term “patterning” refers to a process whereby a molecule is linked only to a portion or region of a support. Typically, such regions are between 1 and 10000 μm, more preferably between 100 and 1000 μm, in width. The length of such regions can vary as needed.

The term “oligonucleotide” as used herein is defined as a molecule comprised of two or more deoxyribonucleotides or ribonucleotides, usually more than three (3), and typically more than ten (10) and up to one hundred (100) or more (although preferably between twenty and thirty). The exact size will depend on many factors, which in turn depends on the ultimate function or use of the oligonucleotide. The oligonucleotide may be generated in any manner, including chemical synthesis, DNA replication, reverse transcription, or a combination thereof.

“Hybridization” methods involve the annealing of a complementary sequence to the target nucleic acid (the sequence to be detected). The ability of two polymers of nucleic acid containing complementary sequences to find each other and anneal through base pairing interaction is a well-recognized phenomenon. The initial observations of the “hybridization” process by Marmur and Lane, Proc. Natl. Acad. Sci. USA 46:453 (1960) and Doty et al., Proc. Natl. Acad. Sci. USA 46:461 (1960) have been followed by the refinement of this process into an essential tool of modern biology.

The complement of a nucleic acid sequence as used herein refers to an oligonucleotide which, when aligned with the nucleic acid sequence such that the 5′ end of one sequence is paired with the 3′ end of the other, is in “antiparallel association.” Certain bases not commonly found in natural nucleic acids may be included in the nucleic acids of the present invention and include, for example, inosine and 7-deazaguanine. Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

Stability of a nucleic acid duplex is measured by the melting temperature, or “Tₘ.” The Tₘ of a particular nucleic acid duplex under specified conditions is the temperature at which on average half of the base pairs have disassociated. The equation for calculating the Tₘ of nucleic acids is well known in the art.

A “label” is a detectable marker, such as a dye or radiolabel.

### DESCRIPTION OF THE INVENTION

The present invention relates to the coating of silicon surfaces, including but not limited to coating silicon surfaces with biomolecules such as biotin and avidin. The present invention contemplates coating silicon surfaces with a monolayer of the protein streptavidin. These surfaces can then be used as supports for reactions. In one embodiment, single-stranded biotinylated nucleic acids are bound to the streptavidin, thereby providing a microarray of nucleic acid for hybridization with target nucleic acid.

Because the biotin-streptavidin interaction is both specific and strong (K_{d}=10⁻¹⁵M), biotinylated biomolecules adhere specifically to the space of deposition. Shalon et al. and others have used NHS-modified alkyl chains covalently bound to the silicon dioxide surface as linkers to which is attached amine-modified PCR products. However, the biotin-streptavidin interaction employed in the instant invention is more specific than using NHS/amine chemistry. For example, in the Shalon et al approach, the NH₃+ reactive end can also bind non-specifically to the anionic phosphate backbone of nucleic acids. By contrast, biotin will not bind non-specifically to nucleic acids.

Furthermore, biotin is easily incorporated into oligonucleotides, either 5′ or 3′, and can also be used in designing primers for PCR amplification to obtain large (500-30,000 bp) biotinylated double-stranded DNAs. Both ends of double-stranded DNAs can be labeled with biotin at their 3′ ends using terminal transferase, and 3′ recessed double-stranded ends can be extended using DNA polymerase with one or more biotin-dNTPs.

The description of the invention involves I) design of microarrays using silicon and glass substrates, and II) carrying out reactions on microarrays.

I. Design Of Microarrays

Although there are many formats, materials, and size scales for constructing arrays, the present invention contemplates silicon microfabricated devices as a cost-effective solution. Silicon is the material used for the construction of computing microprocessors and its fabrication technologies have developed at an unprecedented pace over the past 30 years. While this technology was initially applied to making microelectronic devices, the same techniques are currently being used for microarrays.

As a mechanical building material, silicon has well-known fabrication characteristics. The economic attraction of silicon devices is that their associated micromachining technologies are, essentially, photographic reproduction techniques. In these processes, transparent templates or masks containing opaque designs are used to photodefine objects on the surface of the silicon substrate. The patterns on the templates are generated with computer-aided design programs and can delineate structures with line-widths of less than one micron. Once a template is generated, it can be used almost indefinitely to produce identical replicate structures. Consequently, even extremely complex micromachines can be reproduced in mass quantities and at low incremental unit cost—provided that all of the components are compatible with the silicon micromachining process. While other substrates, such as glass or quartz, can use photolithographic methods to construct microfabricated analysis devices, only silicon gives the added advantage of allowing a large variety of electronic components to be fabricated within the same structure.

The principal modern method for fabricating is the so-called planar process. The planar process relies on the unique characteristics of silicon and comprises a complex sequence of manufacturing steps involving deposition, oxidation, photolithography, diffusion and/or ion implantation, and metallization, to fabricate a “layered” integrated circuit device in a silicon substrate. See e.g., W. Miller, U.S. Pat. No. 5,091,328, hereby incorporated by reference.

A. Modifying The Support Surface

Once as silicon device is fabricated, the surface should be prepared in order to permit the immobilization of biotin. As noted above, the surface of the solid support can thought of as comprising three parts: surface-linker-Y, wherein Y is a functional group capable of chemically interacting with X of X-linker-biotin.

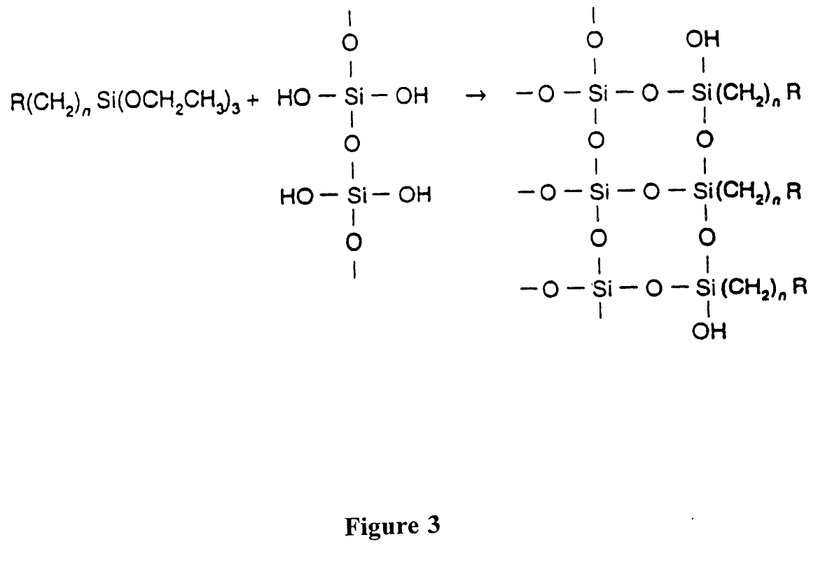

Silanization is a convenient approach to surface modification. See generally, H. H. Weetal, “Preparation of Immobilized Proteins Covalently Coupled Through Silane Coupling Agents to Inorganic Supports,” Applied Biochem. and Biotech. 41:157 (1993). A variety of silanes are available for this purpose, including epoxysilanes, aminosilanes, cyanosilanes, sulfhydrylsilanes, phenylic silanes and glycidoxysilanes, in methoxy, ethoxy and chloro forms. FIG. 3 shows a representative reaction between gamma-aminopropyltriethoxysilane and support material. The ethoxy group reacts with the available silanol residues forming a silioane linkage. In addition, the silane appears to polymerase over the support surface. The product of this particular reaction is ethanol.

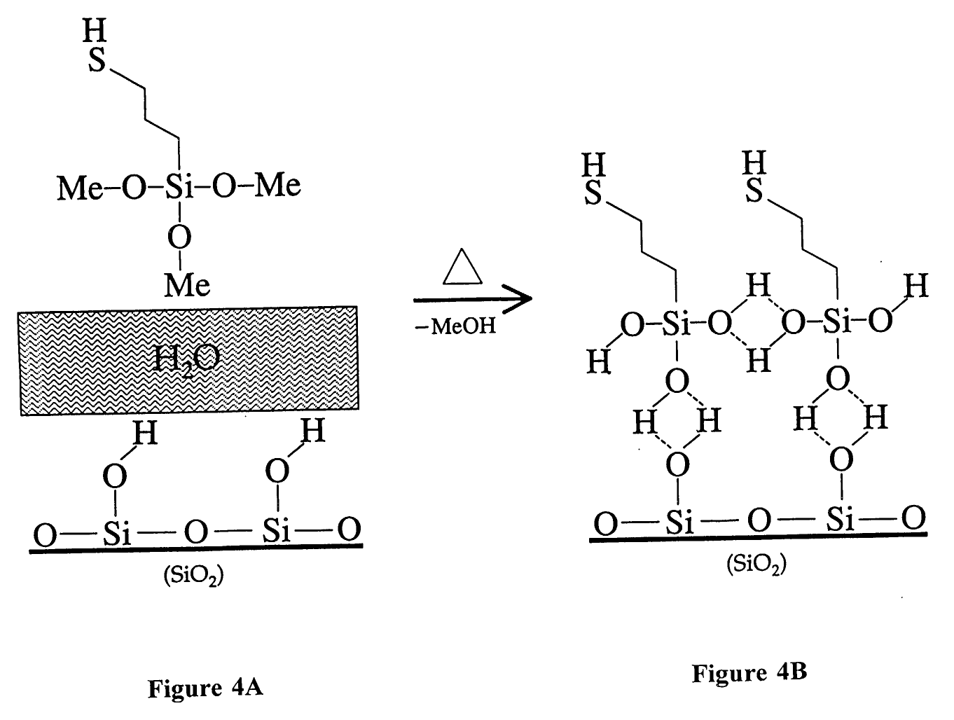

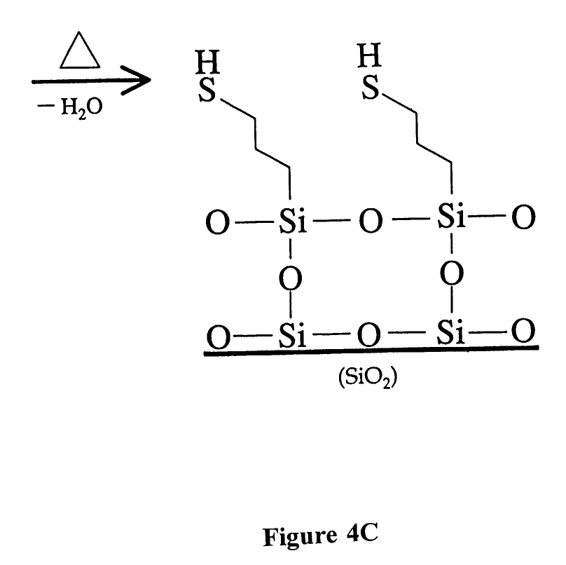

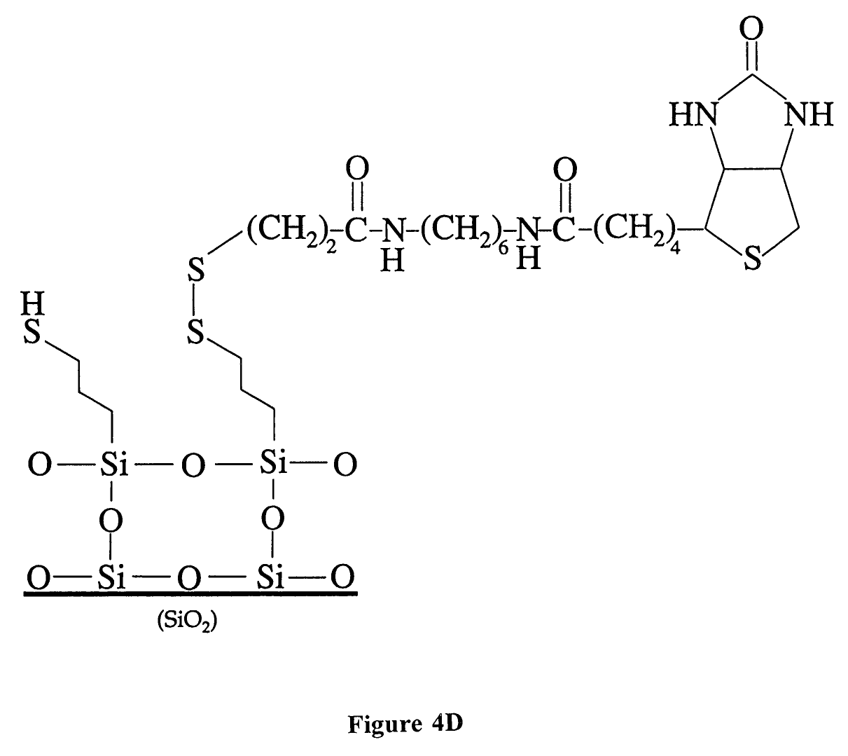

FIGS. 4A, 4B, 4C and 4D show one embodiment of a method for covalent attachment of biotin to silicon dioxide supports. Briefly, silicon dioxide surfaces are cleaned by rinsing with acetone, followed by ethanol, followed by Milli-Q water, then immersed into H₂O₂: NH₄OH:H₂O (1:1:5) heated to 70° C. for 1 hour. The surfaces are rinsed extensively with Milli-Q filtered water and dried under a stream of nitrogen. This cleaning procedure removes (oxidizes) any carbons on the surface and also adds terminal hydroxyls to the silicon dioxide surface. 100 μl of 1% solution of (3-mercaptopropyl)-trimethoxy silane in CHCl₃ (or in any other suitable solvent that can solubilized the silane, such solvents including but not limited to methanol and ethanol) is deposited onto a 144 mm² silicon chip (FIG. 4A), left to evaporate at room temperature, rinsed 3× with CHCl₃, and baked in an oven set to 110-120° C. for 5 hours. The baking process helps to induce evaporation and formation of a stable covalent bond between the trimethoxysilate and the oxide substrate (FIGS. 4B-C). FIG. 4C shows the composition of the substrate after baking—it is highly likely that disulfide formation between adjacent sulfhydryls, so disulfides are reduced by incubating the chips in dithiothreitol or sodium borohydride in buffer (e.g. 0.1M Tris-HCl, 1 mM EDTA, pH 8.0).

Chips are then incubated in 1.5 mM biotin-HPDP in DMF for 1 hour, rinsed extensively with PBS/1 mM EDTA pH 7.6. (FIG. D is the end result). Chips are then incubated with 1 μg/ml streptavidin [or avidin, or NeutrAvidin (Pierce) or any other avidin analog] for 30 minutes then left at 4° C. overnight. Prior to use, chips are rinsed with either PBS or Tris-HCl/EDTA buffers to remove non-specifically bound streptavidins.

B. Patterning

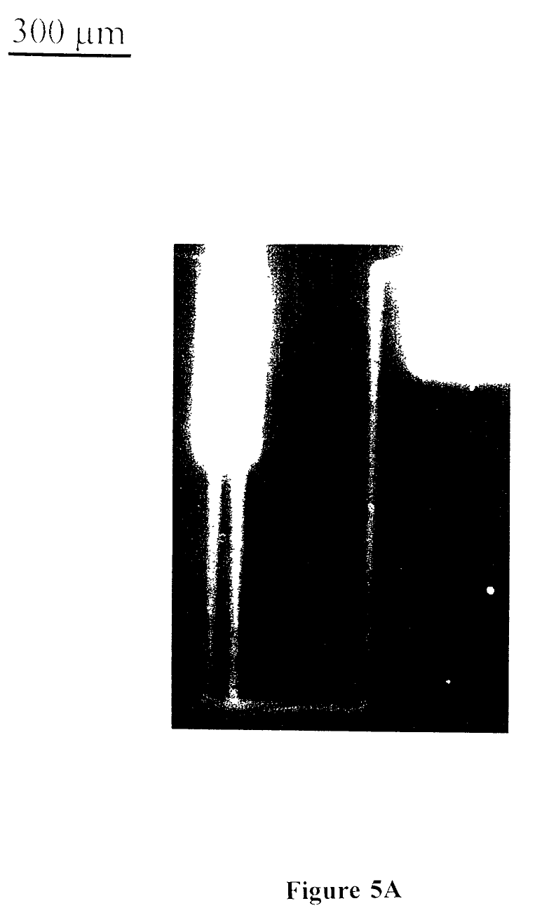

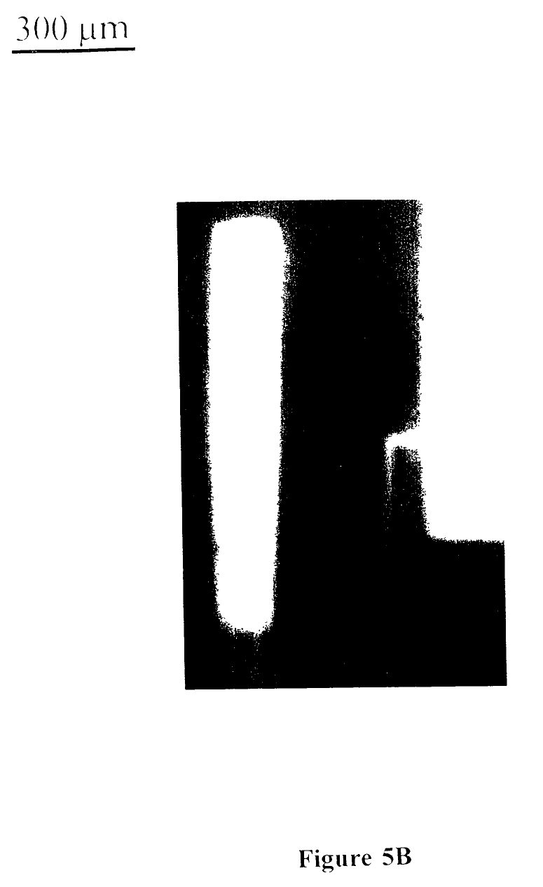

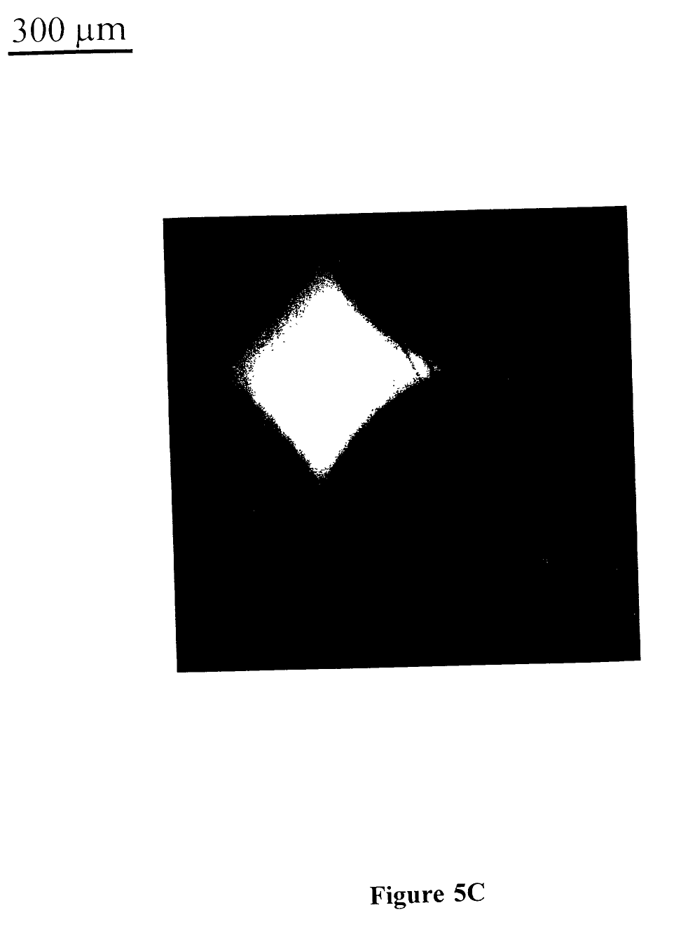

The present invention provides a fast and simple procedure to pattern streptavidin on silicon surfaces. In one embodiment, photoactivatible biotin (e.g. biotin-LC-ASA from Pierce) is taken up to a final concentration of 20 μg/ml in 100% ethanol. 100 μl is spotted onto a 144 mm² silane-silicon chip (silanized). After evaporation, the chip is placed silane side down onto a photomask which contains the negative of the micron-sized features to be patterned. The photomask and the chips are then exposed to 365 nm light for 5 minutes, then extensively rinsed in ethanol. Areas which were exposed to the UV light will contain crosslinks of the biotin-LC-ASA and the siloxane—note that this cross link is non-specific and will react with carbons, hydrogens, sulfurs, etc. within the siloxane (and perhaps even crosslink with itself). The patterned biotin surface is then incubated with 0.1 mg/ml Cy2-streptavidin (Cy2 is a fluorescent label) for 30 minutes, then rinsed extensively with PBS/1 mM EDTA. Micrographs of several patterned streptavidin surfaces are shown in FIG. 5.

Other approaches to patterning are possible. For instance, instead of placing a photomask between a 365 nm UV source and the chip, a laser can be used to ‘write’ features on the chip. The use of a near-field microscope fiber optic source can be used to write features ranging from 50-100 nm, well below the limit of a photomask (approximately 3-5 microns).

II. Reactions

The present invention contemplates coating silicon surfaces with a monolayer of the protein streptavidin. These surfaces can then be used as supports for reactions. It is not intended that the present invention be limited to the nature of the reaction carried out on the microarray. In one embodiment, single-stranded biotinylated nucleic acids are bound to the streptavidin, thereby providing a microarray of nucleic acid for hybridization with target nucleic acid. As demonstrated below, steric factors must be considered when fabricating an array of oligonucleotides. In a preferred embodiment, the biotinylated oligonucleotides attached to streptavidin on the arrays of the present invention are less than fifty nucleotides in length, and more preferably less than 30 nucleotides in length, and still more preferably less than twenty nucleotides in length.

In a preferred embodiment, the arrays of the present invention are used for sequencing nucleic acid, and more specifically sequencing by hybridization (SBH). SBH of a large piece of DNA can be considered a two part process. First, the DNA is fragmented into oligonucleotide N-mers. Thereafter, the N-mers are re-assembled into the complete sequence. Hybridization to probes is used to provide the information for re-assembly. The probes are typically immobilized on a solid support, such as a chip, and arranged in an array.

The number of probes was originally believed to be rigidly set by the requirement for N-mers having all possible sequences. That is to say, since DNA is made up of four (4) bases, the formula was thought to be straightforward: 4ⁿ. Thus, where 8-mers are arrayed, 4⁸ 8-mers are needed on the chip to represent all possible sequences, i.e. 65,536 distinct 8-mers. However, the formula quickly makes the requisite number of probes unwieldy for longer oligos (e.g. approximately 4 million probes are required to represent all 11-mers). Rather than making such a large array, one skilled in the art know that the four bases of DNA can be put in groupings as degenerate probes. For example, where the sequence is characterized as purines (R) and pyrimidines (Y), a 16-mer becomes binary and the formula changes from 4¹⁶ to 2¹⁶ (or 65,536 probes).

U.S. Pat. No. 5,525,464 to Drmanac et al. (hereby incorporated by reference) describes the general SBH technique as well as the concept of reducing probe number. The present invention contemplates using the improved methods and compositions described herein to produce arrays useful in the SBH technique.

Experimental

The following examples serve to illustrate certain preferred embodiments and aspects of the present invention and are not to be construed as limiting the scope thereof.

In the experimental disclosure which follows, the following abbreviations apply: eq (equivalents); μ (micron); M (Molar); μM (micromolar); mM (millimolar); N (Normal); mol (moles); mmol (millimoles); μmol (micromoles); nmol (nanomoles); g (grams); mg (milligrams); μg (micrograms); ng (nanograms); L (liters); ml (milliliters); μl (microliters); cm (centimeters); mm (millimeters); μm (micrometers); nM (nanomolar); ° C. (degrees Centigrade); PBS (phosphate buffered saline); U (units); d(days).

### EXAMPLE 1

In this example, the biotin-binding capacity of streptavidin attached to a silicon support (in the manner described above) was assessed. For this experiment, a microchip was tested, the chip comprising biotin covalently attached to (e.g. immobilized on) a solid support, said solid support comprising silicon dioxide and said biotin non-covalently attached to streptavidin. ¹⁴C-biotin was added to the chip at various concentrations in buffer (Tris-HCl/EDTA) for 2 hours. The chip was then washed with buffer and counts were measured by scintillation counting.

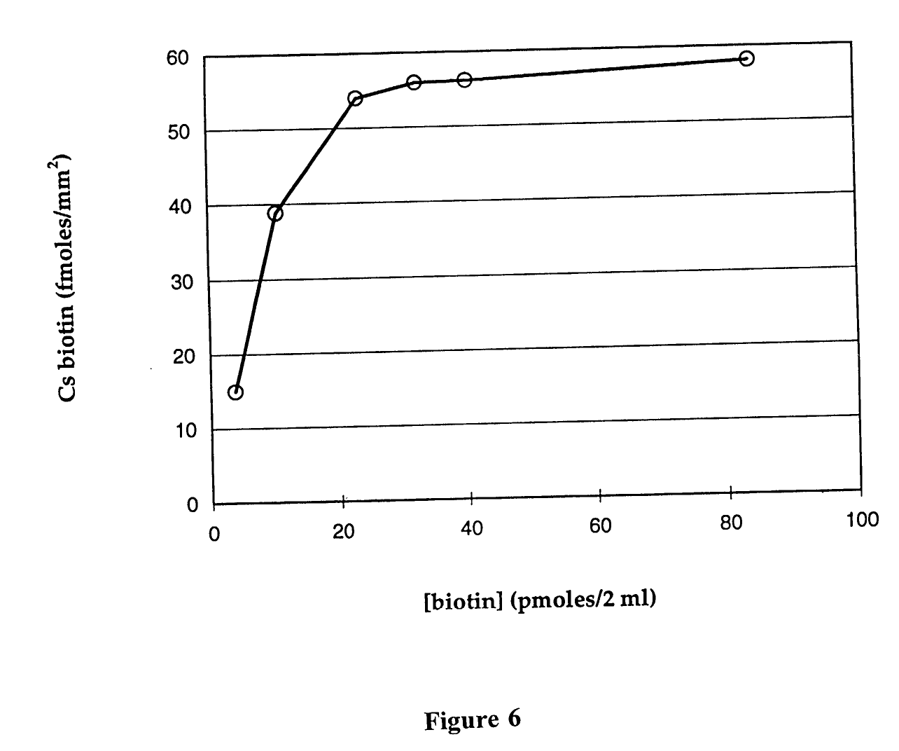

FIG. 6 is a graph showing that free biotin-binding capacity of the streptavidin supports is approximately 55-60 fmoles/mm²—this is about 60% of the maximum theoretical capacity.

### EXAMPLE 2

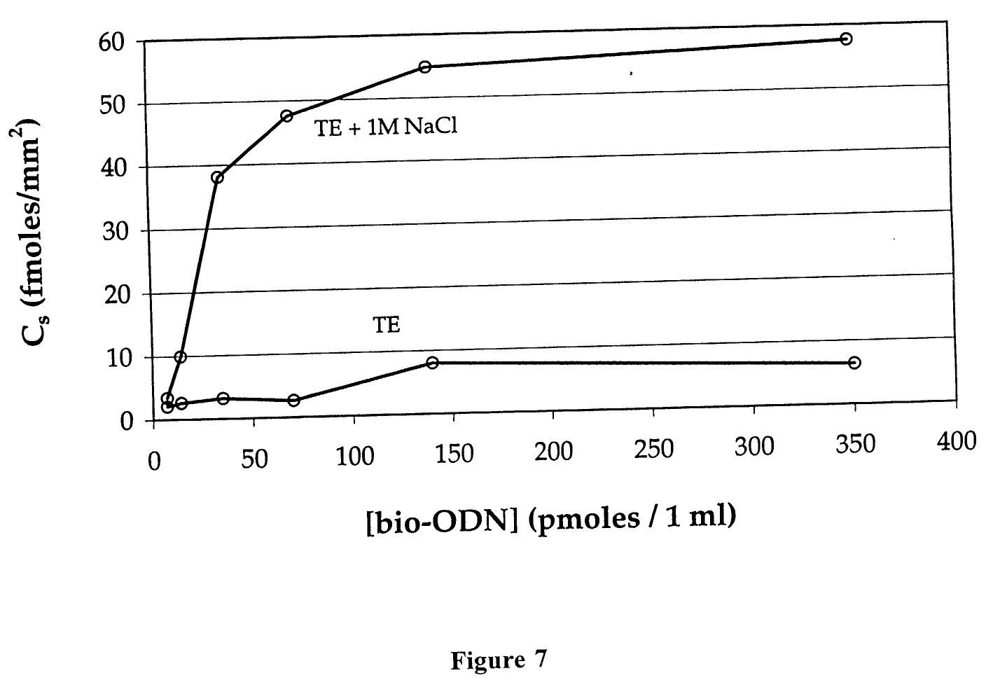

In this example, biotinylated oligonucleotides are attached to streptavidin attached to a silicon support. For this experiment, a chip was tested, the chip comprising biotin covalently attached to (e.g. immobilized on) a solid support, said solid support comprising silicon dioxide and said biotin non-covalently attached to streptavidin. A biotin-labelled 100-mer was reacted in buffer (TE or TE with 1 M NaCl) with the streptavidin. FIG. 7 shows the results. It is clear that oligonucleotides can be adsorbed if there are high salt concentrations present.

### EXAMPLE 3

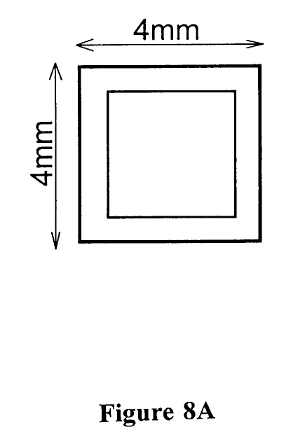

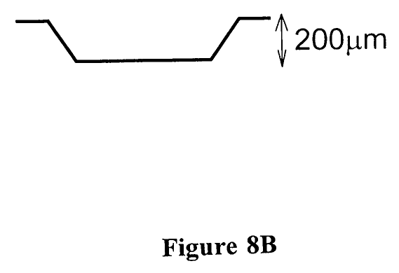

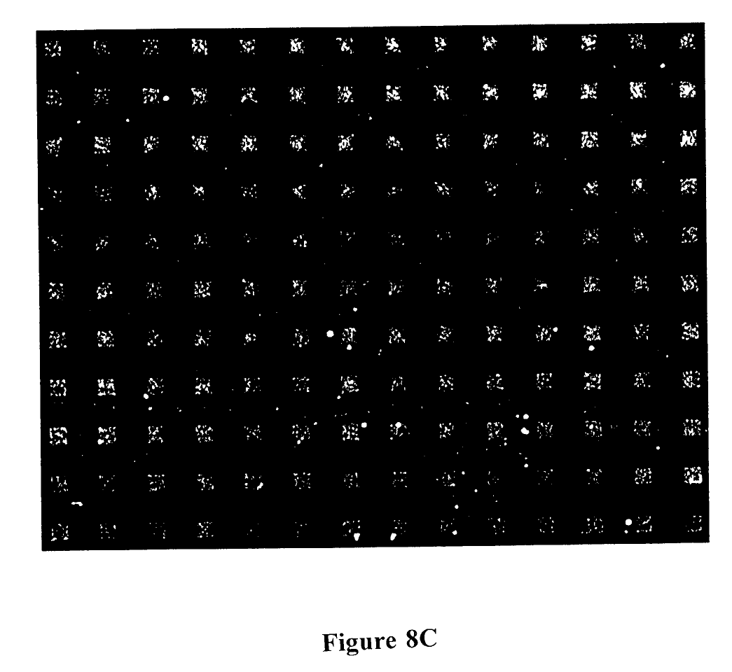

As noted above, biotin (and therefore streptavidin) can be patterned in silicon. In this example, streptavidin is discretely patterned in silicon microwells. FIG. 8A is a top view of a microwell. FIG. 8B is a side view of the well.

The patterning was done as follows. Photoactivatible biotin (e.g. biotin-LC-ASA from Pierce) was taken up to a final concentration of 20 μg/ml in 100% ethanol and was spotted on a silane-silicon chip (silanized). After evaporation, the chip was placed silane side down onto a photomask which contains the negative of the micron-sized features to be patterned. The photomask and the chips were then exposed to 365 nm light for 5 minutes, then extensively rinsed in ethanol. The patterned biotin surface was then incubated with 0.1 mg/ml Cy2-streptavidin for 30 minutes, then rinsed extensively with PBS/1 mM EDTA. A micrograph (FIG. 8C) shows that the streptavidin is patterned in the microwells.

### EXAMPLE 4

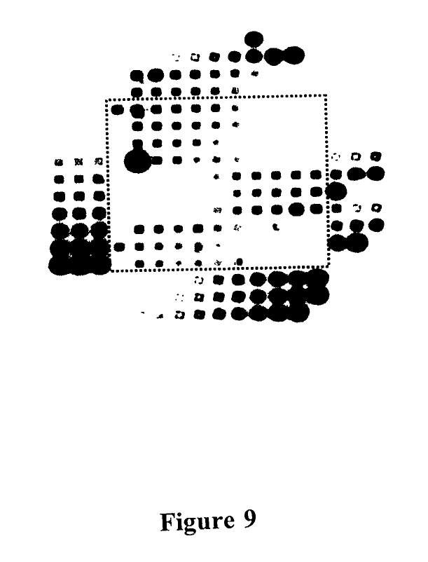

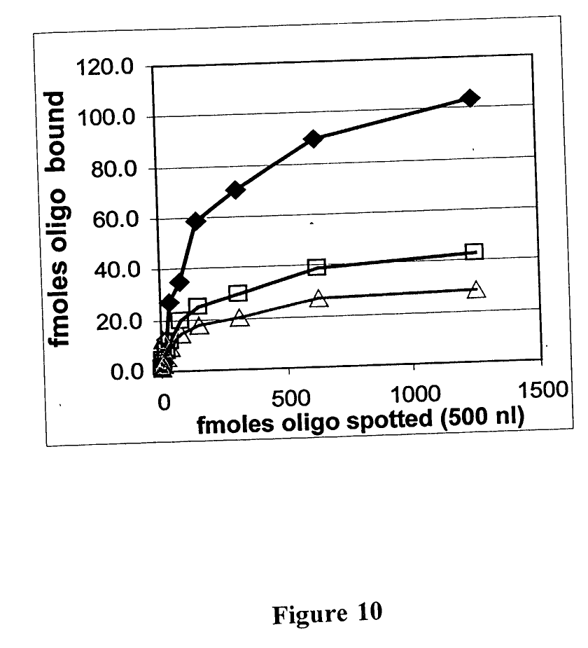

In this example, the patterned microwells of Example 3 are used to capture oligonucleotides. Three ³²P-labeled biotinylated oligonucleotides of different length (a 16-mer, a 52-mer and a 99-mer) were tested by reacting them with the patterned microwells in buffer (TE with 1 M NaCl). FIG. 9 is an autoradiogram showing the results. FIG. 10 is a graph depicting the results in the context of concentration and oligonucleotide length. All of the oligonucleotides show an increase in binding that is dependent on the spotting concentration. However, the size of the oligonucleotide has an impact on the extent of binding, with the 16-mer (solid squares) showing the best binding, followed by the 52-mer (open squares), and lowest level of binding shown by the 99-mer (open diamonds). Clearly, oligonucleotide binding capacity of the wells increases as oligonucleotide length decreases.

### EXAMPLE 5

In this example, a DNA library representing known genetic mutations is arrayed onto high-density streptavidin supports such that each element of the array contains a unique DNA sequence. Each fragment of the DNA library is constructed via PCR, with one of the primers containing a 5′-biotin molecule. After arraying, the entire library onto the streptavidin support, the arrayed library is heat denatured leaving single-stranded fragments of the library bound to the streptavidin surface. Target DNA obtained from blood or tissues are PCR amplified in the presence of fluorescent dinucleotide triphosphates, heat denatured then spotted onto the DNA-chip. Array elements that yield fluorescent signals depict complementary sequences between the immobilized probes and the target sequences.

### EXAMPLE 6

In this example, an antibody library to known antigens is arrayed onto high-density streptavidin supports such that each element of the array contains a unique antibody. Each antibody from the library is produced by a hybridoma cell line. After purification, antibodies are labeled with biotin [according to the manufacturer's (Pierce) protocol for “EZ-Link Biotin-HPDP”] and arrayed onto streptavidin supports. Serum containing target biomolecules are spotted onto the biochip, washed and a pooled collection of secondary fluorescently labeled antibodies (with different binding sites to the antigens) is spotted onto the chip. Array elements that yield fluorescent signals depict antigen-antibody-antigen complexes.

Another variation of an antibody-antigen detection method is to nonspecifically label proteins (assuming the antigen is a protein) with a fluorescent molecule [using the manufacturer's (Molecular Probes) protocol for Texas Red and its Protein Conjugates].

### EXAMPLE 7

The high-density streptavidin supports can be utilized as a purification system for any molecule continuing biotin. A specific DNA fiber is purified from a mixture of different DNA molecules (for example, a particular chromosome, or chromosal fragment of an organism's entire genome) through a strand-displacement interaction between an immobilized (biotinylated) single-stranded probe sequence (100-2,000 bases) and the double-stranded target DNA. The use of recA protein (a protein of E. coli capable of genetic recombination) promotes the strand exchange of single stranded DNA fragments with homologous duplex DNA.

Importantly, the capture method of the present invention permits analysis directly on the array. That is to say, there is no need to release the captured nucleic acid prior to analysis. Rather, the chip can be directly imaged.

From the above description and examples, it should be clear that the present invention provides an improved approach to the manufacture of arrays of biomolecules. Such arrays can be used for a variety of reactions, including but not limited to sequencing of nucleic acids.

1. A method, comprising:
a) providing i) biotin covalently attached to a solid support, said solid support comprising silicon dioxide, and ii) streptavidin; and
b) non-covalently attaching said streptavidin to said biotin, thereby attaching said streptavidin to said solid support.

2. The method of claim 1, further comprises the step c) binding a biotin-linked biomolecule to said streptavidin attached to said solid support.

3. The method of claim 2, wherein said biotin-linked biomolecule is selected from the group consisting of biotin-linked proteins and biotin-linked nucleic acids.

4. A method, comprising:
a) providing i) photoactivatible biotin, ii) a solid support, said solid support comprising silicon dioxide, and iii) a source of light; and
b) bring said photoactivatible biotin in contact with said solid support in the presence of light from said light source, under conditions such that biotin is covalently attach to said solid support.

5. The method of claim 4, further comprising the step c) non-covalently attaching streptavidin to said biotin attached to said solid support to create bound streptavidin.

6. The method of claim 5, further comprises the step d) binding a biotin-linked biomolecule to said bound streptavidin.

7. The method of claim 6, wherein said biotin-linked biomolecule is selected from the group consisting of biotin-linked proteins and biotin-linked nucleic acids.

8. A composition, comprising biotin covalently attached to a solid support, said solid support comprising silicon dioxide and said biotin non-covalently attached to streptavidin.
